(19) United States
(12) Patent Application Publication
(10) Pub. No.: US 2009/0238717 A1
(43) Pub. Date: Sep. 24, 2009

(54) SYSTEMS AND METHODS FOR HEATING A LIQUID FUEL OF A CANDLE

(76) Inventors: Medrith Deborah Nolan, Los Gatos, CA (US); Jere Donald Nolan, Los Gatos, CA (US)

Correspondence Address: THE NOBLITT GROUP, PLLC, 4800 NORTH SCOTTSDALE ROAD, SUITE 6000, SCOTTSDALE, AZ, 85251, US

(21) Appl. No.: 12/410,169

(22) Filed: Mar. 24, 2009

Related U.S. Application Data
(60) Provisional application No. 61/070,695, filed on Mar. 24, 2008.

(51) Int. Cl. A61L 9/02; F23D 3/24; A61L 9/013

(52) U.S. Cl. 422/5; 431/320; 431/323; 422/126

(57) ABSTRACT

Systems and methods for heating a candle fuel generally comprise: a vessel to contain a quantity of a liquid fuel, and a wick system configured to float upon the quantity of the liquid fuel and to transfer heat to the liquid fuel, wherein the wick system comprises a wick and a wick holder. The wick comprises a first wick material configured to support a flame from a combustion and a second wick material, proximate to the first wick material, and configured to absorb combustion products from the combustion. The wick holder is configured to support the wick, such that at least a portion of the wick is immersed in the quantity of the liquid fuel.

### CROSS-REFERENCES TO RELATED APPLICATIONS

This application claims the benefit of U.S. Provisional Patent Application No. 61/070,695, filed Mar. 24, 2008 and incorporates the disclosure of that application by reference.

### FIELD OF INVENTION

The present invention generally concerns candle systems, and more particularly, representative and exemplary embodiments of the present invention generally relate to systems, devices, and methods relating to a wick system that is configured to provide heat to a product, wherein the product simultaneously operates as a fuel for the candle.

### BACKGROUND OF INVENTION

In general, candle systems comprise a fuel source and a wick. The wick is coupled to the fuel source in some manner that provides fuel for the wick to burn, which in turn provides heat and light. Generally, the fuel source is a solid paraffin wax or a liquid lamp oil. Through capillary action or proximate placement, these fuels provide the source for the wick to bum.

To provide for a more aesthetically pleasing candle system, candles comprising wick systems that “float” in a liquid medium have witnessed great popularity. Moreover, candle systems that provide therapeutic qualities, e.g. aromatherapy, have also witnessed great popularity. As such, an aesthetically pleasing, floating wick system that burns and provides heat in a manner that overcomes the limitations of prior art floating wick systems is disclosed.

### SUMMARY OF THE INVENTION

In various representative aspects, the present invention comprises a candle system that comprises a wick system, which floats in a liquid fuel for the candle, and the wick system is configured to conduct heat to the candle's liquid fuel. The liquid fuel comprises a substantially organic material and in addition to functioning as the fuel for the wick system, the organic material may be used, in one example, as a therapeutic skin medicament, but in other embodiments the organic material may be used as a food condiment or an aromatherapy composition for inhalation.

In an embodiment, systems and methods for a candle fuel heating system may generally comprise, a vessel to contain a quantity of a liquid fuel, and a wick system configured to float upon the quantity of the liquid fuel and to transfer heat to the liquid fuel, wherein the wick system may comprise a wick and a wick holder. The wick may comprise a first wick material configured to support a flame from a combustion, and the wick may comprise a second wick material, proximate to the first wick material, and configured to absorb combustion products from the combustion. The wick may further comprise a metallic sleeve to support the first and second wick material, as well as conduct heat from the flame to the liquid fuel. The wick holder may be configured to support the wick, such that at least a portion of the wick may be immersed in the quantity of the liquid fuel.

### BRIEF DESCRIPTION OF THE DRAWINGS

A more complete understanding of the present invention may be derived by referring to the detailed description and claims when considered in connection with the following illustrative figures. In the following figures, like reference numbers refer to similar elements and steps throughout the figures.

FIG. 1 representatively illustrates a candle system, according to an exemplary embodiment of the present invention;

FIG. 2 representatively illustrates an exploded view of a wick, according to an exemplary embodiment of the present invention;

FIG. 3 representatively illustrates the wick, according to the exemplary embodiment of the present invention; and

FIG. 4 representatively illustrates a wick system, according to an exemplary embodiment of the present invention.

Elements and steps in the figures are illustrated for simplicity and clarity and have not necessarily been rendered according to any particular sequence. For example, steps that may be performed concurrently or in different order are illustrated in the figures to help to improve understanding of embodiments of the present invention.

### DETAILED DESCRIPTION OF EXEMPLARY EMBODIMENTS

The present invention may be described herein in terms of functional block components and various processing steps. Such functional blocks may be realized by any number of hardware and/or software components configured to perform the specified functions and achieve the various results. For example, the present invention may employ various wicks and wick holders, e.g., compound wicks, refractory wicks, heat conductive wicks, decorative wicks, buoyant wick holders, glass wick holders, and the like, which may carry out a variety of functions. In addition, the present invention may be practiced in conjunction with any number of environments for using a candle system, and the system described is merely one exemplary application for the invention. Further, the present invention may employ any number of conventional techniques for burning a candle, heating a product, providing therapeutic environmental settings, and the like.

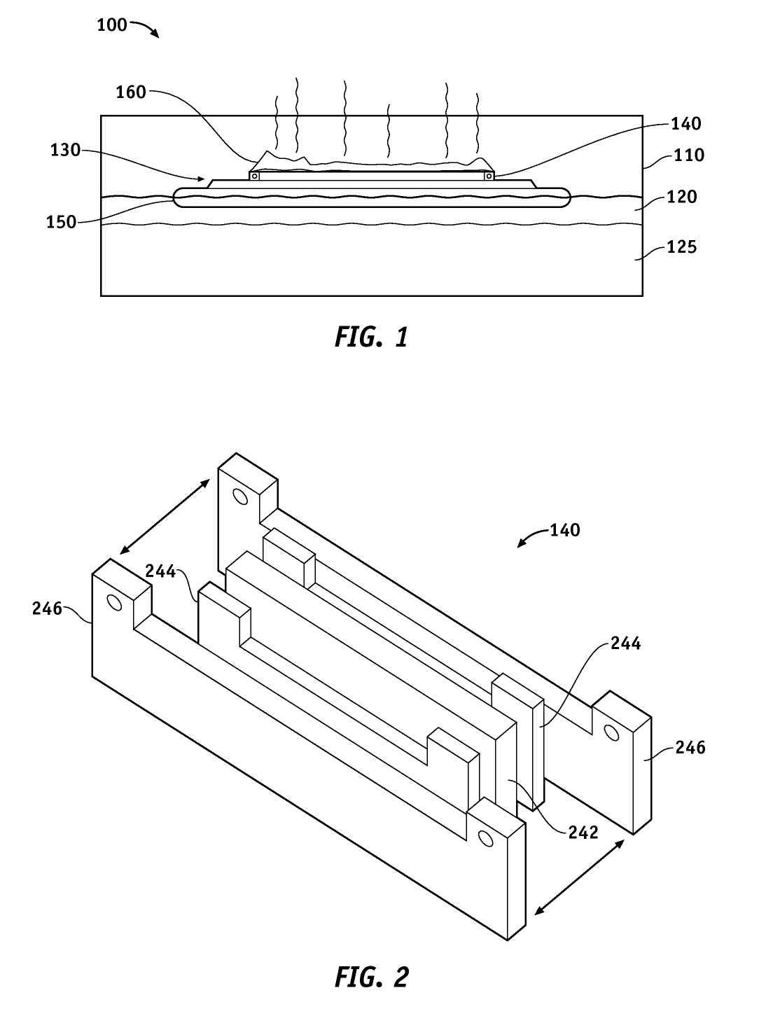

Various representative implementations of the present invention may be applied to any system for operating a candle system and/or heating a candle fuel. Certain representative implementations may include, for example: providing heat and light, providing a decorative, aesthetically pleasing object, heating a product, providing therapy, etc. Systems and methods for heating a candle fuel may operate in conjunction with a candle fuel heating system 100. Referring now to FIG. 1, FIG. 1 shows an embodiment of the present invention. In accordance with an exemplary embodiment, the candle fuel heating system 100 may comprise, a vessel 110 to contain a quantity of a liquid fuel 120, and a wick system 130 configured to float upon the quantity of the liquid fuel 120. The wick system 130 may comprise a wick 140 and a wick holder 150. In an exemplary embodiment, the wick 140 supports a flame 160 from combustion of the ignited liquid fuel 120. The wick 140 may be further configured to transmit heat, e.g. via conduction, from the flame 160 to the liquid fuel 120 so as to warm the liquid fuel 120. Once warm, the liquid fuel 120 may be used by a user. In one example, the warmed liquid fuel 120 may be used as a skin medicament, but in other examples, the warmed liquid fuel 120 may be used as a food condiment or comprise an aromatherapy composition, however, the candle fuel heating system 100 is not limited in this regard.

In accordance with an exemplary embodiment of the present invention, the system 100 may comprise the vessel 110 to contain a quantity of the liquid fuel 120. The vessel 110 may comprise any configuration to adequately, but more importantly, safely contain the quantity of the liquid fuel 120. In one embodiment, the vessel 110 may comprise a clear glass configuration, such that the light from the flame 160 illuminates the entirety of the candle fuel heating system 100 to provide for a superior, aesthetically pleasing object. In an embodiment, the clear vessel 110, comprising a clear liquid fuel 120 and a clear wick system 130, promotes a unique, “fire floating on water” appearance. In other embodiments, the vessel 10 may comprise colored glass, frosted glass, opaque or semi-opaque glass, etched glass, mosaic glass, etc. In yet other embodiments, the vessel 110 may comprise of materials other than glass, for example, synthetic materials, polymeric materials, natural materials, and/or combinations thereof. In the exemplary embodiment shown in FIG. 1, the vessel 110 comprises a square or rectangular configuration, but other shapes, either regular or irregular, comprising various heights, widths, depths, diameters, perimeters, and the like may be used.

In accordance with an exemplary embodiment, the candle fuel heating system 100 may comprise a liquid fuel 120 that may combust when ignited to provide the flame 160 via the wick 140. In an embodiment, the liquid fuel 120 comprises an all natural organic oil that can operate not only as a fuel, but also as a usable consumer product. For example, the liquid fuel 120 may comprise of a fractured coconut oil. The coconut oil is combustible, yet it may also be used as a skin medicament, i.e. a moisturizer. Thus, as the candle system 100 operates, the coconut oil is heated and a user can dip into the oil to use, for example, on their skin. In another embodiment, the liquid fuel 120 may comprise enriched olive oil, wherein the heated olive oil may be used as a food condiment. The present invention is not limited by these examples, and any other embodiment that may benefit from heated natural oil or other materials is contemplated by this disclosure.

It should be noted that candle systems, in general, do not use natural organic oils as fuels because combustion products, such as carbon, can quickly build up and clog the wick material, thereby preventing the candle from operating effectively. As discussed further below, the present invention overcomes this limitation such that the benefits of using natural oils that are both combustible and/or consumable may be used.

In accordance with an exemplary embodiment, the liquid fuel 120 may comprise other combustible materials other than natural oils, for example, kerosene, lamp oil, etc. Moreover, exemplary embodiments may comprise other combustible, consumable materials other than a natural oil. Although not necessary, many of the natural oils are preferred over typical candle fuels because of the high flash point of the natural oils. Because of the nature of the candle systems described, which rely upon a bath of fuel oil for the wick system to float, a fuel having a low flash point may be hazardous, e.g. the entire bath surface could ignite. By using the natural oils that have a substantially higher flash point, such hazard is avoided.

In accordance with an exemplary embodiment and with continued reference to FIG. 1, the liquid fuel 120 may be positioned on top of a layer of water 125 or any other non-combustible element. In this embodiment, the water 125 may act as a safety barrier for the candle fuel heating system 100. For example, once the wick system 130 consumes all of the oil 120, the wick system 130 will extinguish itself once it reaches the water layer 125. Furthermore, a user may control the duration the candle fuel heating system 100 operates merely by adjusting the oil level 120 placed on top of the water layer 125. As a further benefit, the water layer 125 may also act as a quick extinguishing mechanism for a user. If the user wishes to quickly extinguish the flame 160, the user only needs to push the wick holder 150, comprising the wick 140, down to the water layer 125.

In accordance with an exemplary embodiment, FIG. 2 representatively illustrates an exploded view of the wick 140. The wick 140 may comprise at least a first wick material 242 configured to support the flame 160 from a combustion of ignited fuel oil 120. The wick 140 may also comprise a second wick material 244 positioned proximate and/or suitably coupled to the first wick material 242 and, in an embodiment, may be configured to absorb combustion by-products, for example, carbon. The wick 140 may further comprise a wick sleeve 246 configured to support the first wick material 242 and the second wick material 244, facilitate heat transfer from the ignited flame 160 to the liquid fuel 120, as well as deter any absorbed by-products from releasing back into the liquid fuel bath.

In accordance with an exemplary embodiment, and as best seen by the FIGS. 1-4, the wick 140 may comprise a rigid or semi-rigid, straight, substantially horizontal configuration. This configuration provides mass appeal by contributing to the overall aesthetics of the candle fuel heating system 100. Moreover, the wick 140 in addition to comprising a substantially straight, horizontal configuration as shown, may be configured, as a benefit of a rigid embodiment, to sustain other decorative configurations, such as words, numbers, shapes, figures, combinations thereof, and any other free form shapes.

In an embodiment and in addition to the aesthetic appeal, the substantially horizontal wick 140 may also provide for a more efficient manner to heat the liquid fuel 120, i.e. a greater quantity of flame may be supported to heat the liquid fuel 120.

Among various embodiments, the first wick material 242 may comprise any material that can act in a wicking manner to supply the liquid fuel 120 to promote the combustion and the flame 160. In an embodiment, the first wick material 242 may comprise a non-flammable and/or heat conducting ceramic fiber material, for example, a TaoFibre® Ceramic Fiber Paper, but other materials, for example refractory materials such as, alumina, silica, zirconia, magnesia, combinations thereof, and the like may be used. Although non-flammable, the ceramic fiber material, through capillary action, was found to adequately support the combustion and the flame 160.

In accordance with an embodiment, the first wick material 242 as well as the second wick material 244 described below, because of their fibrous nature are at least partially porous such that liquid additives could be added to the materials. For example, an aromatherapy or air freshener could be added and as the wick bums, the additives would be released into the surrounding environment.

The following Summary Table I illustrates compositions of various other grades of exemplary ceramic fiber materials that may be used. Other candle systems in the prior art have attempted to use such ceramic fibers, but have achieved only limited success. Using these ceramic fiber materials as candle wicks, the fibers can become clogged with a build-up of carbon from the combustion. To overcome this limitation, the present invention may further comprise the second wick material 244.

SUMMARY TABLE I

| | Standard | | Zirconium |
|---|---|---|---|
| Grade | Grade | Premium Grade | Grade |
| Al₂O₃ | 45-46% | 47-49% | 39-40% |
| SiO₂ | 51-52% | 50-52% | 42-43% |
| ZrO₂ | — | — | 15-17% |
| Fe₂O₃ | <1.0% | 0.2% | 0.2% |
| K₂O + Na₂O | ≦0.5% | 0.2% | 0.2% |
| LOI | <10% | <10% | <1 |

In accordance with an exemplary embodiment of the present invention, the wick 140 may comprise the second wick material 244, which may be suitably coupled to or positioned proximate to the first wick material 242. The second wick material may function as a combustion by-product sink for components of the combustion, for example, carbon. As mentioned briefly, wicking materials such as the ceramic fibrous types discussed are generally not used as wicks for candle systems. They have a tendency to clog with combustion by-products thereby limiting their effective operation because of carbon build-up. To alleviate this, the present invention comprises a material to absorb the by-products, which greatly enhances the operational use of the ceramic fiber wick. In one example, the second wick material 244 may comprise a high alpha cellulose material, but almost any material that can be sacrificially coupled to or positioned proximate to the first wick material 242 to absorb combustion by-products, may be used. Other paper examples may comprise blotting type paper, bond paper, construction paper, cotton paper, woven and/or coated paper, and the like. It should be appreciated that the second wick material 244, by absorbing combustion products, further promotes the clean, clear look of the system 100, i.e. the second wick material 244 may substantially prevent or deter carbon and/or other by-products from leaching into the liquid fuel 120, thereby clouding the liquid fuel 120.

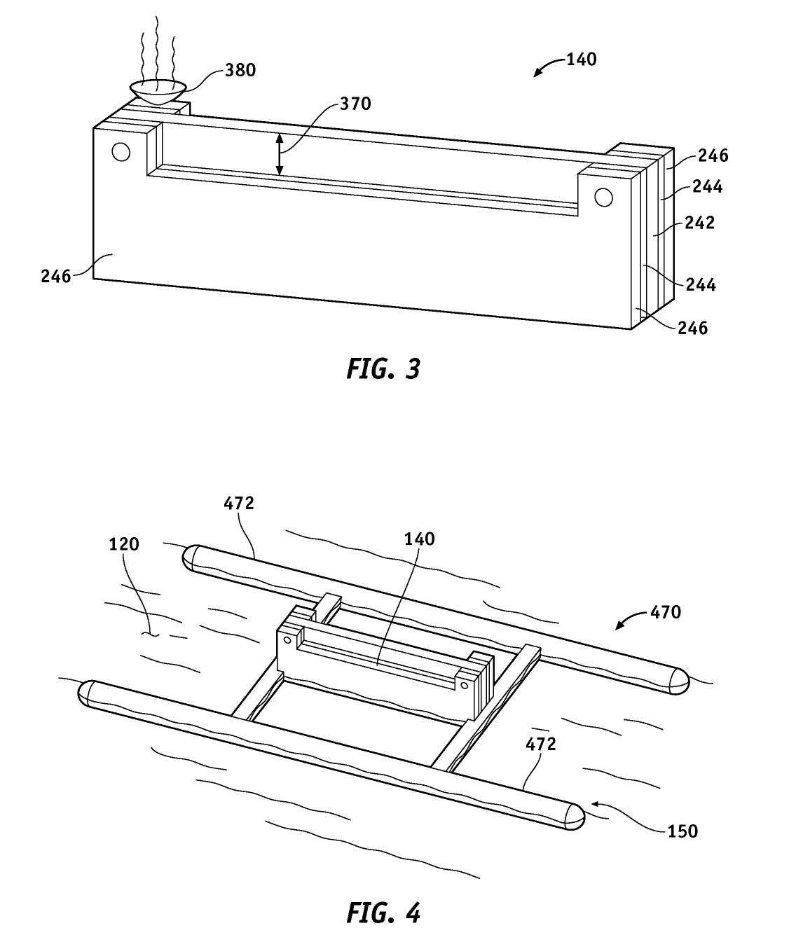

In accordance with an exemplary embodiment of the present invention, the wick 140 may comprise the sleeve 246. In some embodiments, the first wick material 242 as well as the second wick material 244, may comprise of a rigid configuration, but in some embodiments, the first and second wick materials may comprise less rigid or flaccid configurations and therefore, rely upon the sleeve 246 for support The sleeve 246 may be configured to support the first wick material 242 and/or the second wick material 244. The sleeve 246 may comprise substantially parallel plates that fasten together to sandwich the first and second wick materials together, as more completely shown by FIG. 3. In some embodiments the sleeve 246 may comprise other configurations to support the first and second wick materials. For example, the sleeve 246 may comprise of a single element that is folded in half such that the first and second wick materials can be positioned within the crease of the fold. Also, in such a folded embodiment, the crease may be further perforated to allow the liquid fuel 120 to saturate the wick materials once immersed in the liquid fuel 120. In accordance with an embodiment, the sleeve 246 may comprise of a metallic material to conduct heat from the flame 160 to the liquid fuel 120 so as to warm the liquid fuel 120. It should be appreciated that the sleeve 246 may support the first and second wick materials so as to promote capillary action, which may provide one mechanism for wetting the wick 140 elements when immersed in the liquid fuel 120.

In accordance with an exemplary embodiment, the sleeve 246 may also deter the transmission, i.e. leakage, of combustion by-products into the liquid fuel 120, wherein transmission of the by-products into the liquid fuel 120 would cloud the aesthetically pleasing, clear, fuel oil bath. Whereas the second wick material 244 described above may prevent by-products from clogging the first wick material 242, the sleeve 256, by substantially encasing the first and second wick materials, may prevent the by-product absorbing second wick material 244 from allowing any absorbed by-products from leaching into the bath of the liquid fuel 120.

In accordance with yet another exemplary embodiment, the sleeve 246 may also control how far from the surface of the liquid fuel 120 the combustion occurs, i.e. how high above the bath surface the flame burns. As mentioned briefly, the present invention provides for the appearance of a “floating flame.” In an embodiment, by adjusting the dimensions of the sleeve 256, the flame can be configured to bum at varying heights above the surface of the liquid fuel 120 bath.

In accordance with an exemplary embodiment and with reference to FIG. 3, the wick 140 may be configured so that the flame 160 characteristics may be controlled, for example, a height of the flame 160. In an embodiment, the sleeve 246 may comprise of a notched configuration such that a portion of the first wick material is revealed, for example, a revealed portion 370. By adjusting the amount of the revealed portion 370, the flame height, and thus the flame intensity, can be controlled.

In accordance with an exemplary embodiment, the wick 140 may comprise of a disposable type wick that may be disposable after a certain duration, for example, after 8-12 hours of use. In some embodiments, only particular elements of the wick 140 may be disposable. For example, instead of replacing the entire wick 140 and/or wick holder 150, a user may merely replace the first wick material 242 and/or the second wick material 244 that have become saturated with combustion by-products, worn from use, and the like.

In accordance with an exemplary embodiment, the wick system 130 may comprise the wick holder 150. As exemplary illustrated by FIG. 4, the wick holder 150 may comprise a support system to support the wick 140, as well as provide a mechanism to allow the wick 140 to float upon the liquid fuel 120. In an embodiment, the wick holder 150 may comprise a basic frame 470. The frame 470 may further comprise floats 472 that act as buoyant mechanisms for the frame 470.

In an embodiment, the floats 472 may comprise air filled chambers comprising a fixed and/or an adjustable volume to provide buoyancy for the frame 470 yet also allow the wick 146 to be at least partially immersed in the liquid fuel 120.

In an embodiment of the present invention, the frame 470 may also be configured to provide spacing from a side wall of the vessel 110, thereby preventing the flame 160 from excessively heating any side wall of the vessel 110 as the frame 470 floats about. As can be seen by the configuration of the frame 470 in FIG. 4, the wick 140 may be positioned at substantially the center, or at least an interior portion, of the frame 470, so that as the frame 470 floats on the liquid fuel 120 the floats 472 will encounter any side walls of the vessel 110, thereby preventing the wick 140 from encountering the side walls as described. Moreover, the frame 470 may comprise a size such that the frame 470 fits into a fuel bath comprising an area configure to adequately provide for the frame 470 to float about the bath surface to heat the fuel bath to a safe level given the size of the wick and the flame it supports.

With continued reference to FIG. 4, the wick 140 may be substantially supported by the frame 470 such that the wick may be positioned substantially normal to a surface of the liquid fuel 120. In an embodiment the wick 140 may be secured to the frame 470 by slots so that each end of the wick 140 can be secured therein, but in other embodiments any securing mechanism may be used, such as, glues, epoxies, screws, clips, pins, and the like.

In accordance with an exemplary embodiment, the frame 470 may comprise a clear borosilicate glass material that compliments the aesthetics of the entire candle fuel heating system 100. In other embodiments, other configurations of frame 470 comprising other materials may be used. For example, instead of the floats 472 comprising air filled chambers, the floats may comprise buoyant materials, such as wood, cork, etc. Also, instead of the frame 470 comprising clear borosilicate glass, the frame 470 may comprise a plexi-glass frame, a wood frame, and the like. Moreover, other frame constructions and/or configurations may be used.

In accordance with an exemplary embodiment of the present invention, the candle fuel heating system 100 may comprise complimentary elements that can accentuate any therapeutic qualities the system 100 may provide. In one embodiment, the wick 140 may further comprise a crucible 380, FIG. 3, to contain an aromatherapy composition. As the metallic sleeve 246 is heated by the flame 160, the sleeve 246 conducts at least a portion of the heat to the crucible 380 to heat any contents. This is merely one example of a complimentary element and a myriad of complimentary elements could similarly be used.

In accordance with an exemplary embodiment of a candle fuel heating system 100, a method for using a candle fuel heating system 100 may comprise: filling a vessel with a quantity of a liquid fuel; placing a wick system upon a surface of the liquid fuel; lighting a wick from the wick system to initiate a combustion to produce a flame; and heating the quantity of the liquid fuel by the wick system.

In an exemplary embodiment and similar to the system components described, the wick system may be configured to float upon the liquid fuel and to transfer heat to the liquid fuel wherein, the wick system comprises a wick and a wick holder. The wick may comprises a first wick material configured to support the flame from the combustion, and the wick may comprise a second wick material suitably coupled or proximate to the first wick material and configured to absorb combustion products from the combustion. In an embodiment, the wick holder may be configured to support the wick, such that at least a first portion of the wick is immersed in the quantity of the liquid fuel, and at least a second portion of the wick resides above a surface of the quantity of the liquid fuel. The candle fuel heating method is not limited in this regard, and other methods are contemplated by this disclosure. Although a particular order of actions is disclosed, these actions can be performed in other temporal sequences. For example, two or more actions can be performed sequentially, concurrently, or simultaneously.

Additional examples of such changes have been given in the foregoing description. Accordingly, the disclosure of embodiments of the candle fuel heating system and method is intended to be illustrative of the scope of the candle fuel heating system and method and is not intended to be limiting. For example, in one embodiment, the candle fuel heating system may comprise one or more features of FIG. 1 with or without the features described with reference to FIGS. 2-4. Similarly, in different embodiments, the candle fuel heating system may comprise one or more of the features of FIG. 2 with or without the features of FIGS. 1 and 3-4, the candle fuel heating system may comprise one or more of the features of FIG. 3 with or without the features of FIGS. 1-2 and 4, and the candle fuel heating system may comprise one or more of the features of FIG. 4 with or without the features of FIGS. 1-3.

In the foregoing specification, the invention has been described with reference to specific exemplary embodiments. Various modifications and changes may be made, however, without departing from the scope of the present invention as set forth in the claims. The specification and figures are illustrative, rather than restrictive, and modifications are intended to be included within the scope of the present invention. Accordingly, the scope of the invention should be determined by the claims and their legal equivalents rather than by merely the examples described.

For example, the steps recited in any method or process claims may be executed in any order and are not limited to the specific order presented in the claims. Additionally, the components and/or elements recited in any apparatus claims may be assembled or otherwise operationally configured in a variety of permutations and are accordingly not limited to the specific configuration recited in the claims.

Benefits, other advantages and solutions to problems have been described above with regard to particular embodiments; however, any benefit, advantage, solution to problem or any element that may cause any particular benefit, advantage or solution to occur or to become more pronounced are not to be construed as critical, required or essential features or components of any or all the claims.

As used herein, the terms “comprise”, “comprises”, “comprising”, “having”, “including”, “includes” or any variation thereof, are intended to reference a non-exclusive inclusion, such that a process, method, article, composition or apparatus that comprises a list of elements does not include only those elements recited, but may also include other elements not expressly listed or inherent to such process, method, article, composition or apparatus. Other combinations and/or modifications of the above-described structures, arrangements, applications, proportions, elements, materials or components used in the practice of the present invention, in addition to those not specifically recited, may be varied or otherwise particularly adapted to specific environments, manufacturing specifications, design parameters or other operating requirements without departing from the general principles of the same.

1. A candle system comprising:
a vessel to contain a quantity of a liquid fuel; and
a wick system configured to float upon the quantity of the liquid fuel and to transfer heat to the liquid fuel;
wherein,
the wick system comprises a wick and a wick holder
wherein,
the wick comprises a first wick material configured to support a flame from a combustion, and a second wick material proximate to the first wick material and configured to absorb combustion products from the combustion; and
the wick holder is configured to support the wick, such that at least a portion of the wick is immersed in the quantity of the liquid fuel.

2. A candle system according to claim 1, wherein the liquid fuel comprises an organic oil.

3. A candle system according to claim 2, wherein the organic oil comprises a fractionated coconut oil.

4. A candle system according to claim 1, wherein the first wick material comprises a refractory material.

5. A candle system according to claim 1, wherein the first wick material comprises a heat conductive material.

6. A candle system according to claim 1, wherein the wick further comprises a sleeve configured to support at least a portion of the first wick material and at least a portion of the second wick material.

7. A candle system according to claim 6, wherein the sleeve comprises a metallic material configured to conduct heat from the flame to the liquid fuel.

8. A candle system according to claim 1, wherein the wick holder is configured to maintain the wick spaced apart from a wall of the vessel.

9. A candle system according to claim 1, wherein the wick comprises a substantially horizontal configuration and the wick holder maintains the substantially horizontal wick in a normal configuration with respect to a surface of the liquid fuel.

10. A wick system comprising:
a wick and a wick holder:
wherein,
the wick holder is configured to support the wick, and
wherein,
the wick comprises a substantially horizontal configuration and the wick comprises a first refractory material configured to support a flame from a combustion.

11. A wick system according to claim 10, further comprising:
a second wick material suitably coupled to the first refractory material and configured to absorb carbon products from the combustion; and
a metallic sleeve configured to support the first refractory material and the second wick material, and conduct heat from the flame to a liquid fuel.

12. A wick system according to claim 10, wherein the wick holder comprises a frame comprising a first slot to support a first end of the horizontal wick and a second slot to support a second end of the horizontal wick.

13. A wick system according to claim 12, wherein the frame comprises air chambers comprising a volume configured to maintain a bouncy of the frame in a quantity of the liquid fuel, such that at least a portion of the wick is submerged in the liquid fuel.

14. A wick system according to claim 10, wherein the first refractory material comprises
at least one of an alumina, a silica, a zirconia, and a magnesia.

15. A method for using a therapeutic candle system comprising:
filling a vessel with a quantity of a liquid fuel;
placing a wick system upon a surface of the liquid fuel, wherein the wick system is configured to float upon the liquid fuel and to transfer heat to the liquid fuel;
wherein,
the wick system comprises a wick and a wick holder
wherein,
the wick comprises a first wick material configured to support a flame from a combustion, and a second wick material suitably coupled to the first wick material and configured to absorb combustion products from the combustion; and
the wick holder is configured to support the wick, such that at least a first portion of the wick is immersed in the quantity of the liquid fuel, and at least a second portion of the wick resides above a surface of the quantity of the liquid fuel;
lighting the wick to initiate the combustion to produce the flame; and
heating the quantity of the liquid-fuel by the wick system.

16. A method according to claim 15, wherein filling the vessel with the quantity of the liquid fuel comprises filling the vessel with a fractionated coconut oil.

17. A method according to claim 15, wherein placing the wick system comprises placing the wick system comprising the first wick material to comprise a refractory material.

18. A method according to claim 15, wherein placing the wick system comprises placing the wick system to comprise the wick holder further comprising a sleeve configured to support at least a portion of the first wick material and at least a portion of the second wick material.

19. A method according to claim 18, wherein the sleeve comprises a metallic material configured to conduct heat from the flame to the liquid fuel.

20. A method according to claim 15, wherein placing the wick system comprises placing the wick system to comprise the wick which comprises a substantially horizontal configuration and wherein the wick holder maintains the substantially horizontal wick in a normal configuration with respect to the surface.
